(19) United States
(12) Patent Application Publication
(10) Pub. No.: US 2005/0113655 A1
(43) Pub. Date: May 26, 2005

(54) Wireless pulse oximeter configured for web serving, remote patient monitoring and method of operation

(76) Inventor: Drue A. Hull, Layton, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 10/723,558

(22) Filed: Nov. 26, 2003

(51) Int. Cl. A61B 5/00; A61B 10/00; G06F 17/00

(52) U.S. Cl. 600/323; 600/324; 128/903; 128/920

(57) ABSTRACT

An embodiment of a wireless pulse oximeter according to the present invention may include a wireless sensor input for receiving raw pulse oximetry data and a processor in communication with the wireless sensor. The processor may be configured for processing the raw pulse oximetry data to obtain processed pulse oximetry data and further configured to generate a Web page for displaying the processed pulse oximetry data. The wireless pulse oximeter may further include a wireless transceiver in communication with the processor and configured for communicating oximetry information including any of the raw pulse oximetry data, the processed pulse oximetry data and the Web page. A system for remote patient monitoring and a method for wirelessly transmitting pulse oximetry data are also disclosed.

### FIELD OF THE INVENTION

The present invention relates generally to medical instruments for measuring bodily parameter useful for diagnosing a patient's condition. More particularly, the present invention relates a wireless pulse oximeter capable of being configured as a Web server allowing remote patient monitoring and method of operation.

### BACKGROUND OF THE INVENTION

Physicians and other health care professionals make assessment and treatment decisions based on a patient's vital signs. Traditionally, this involves measuring a patient's temperature, blood pressure, respiratory rate, and heart rate. Over the last two decades, oxygen saturation level and heart rate have become increasingly used as a measure of patient status and as a basis for deciding treatment. For these reasons, pulse oximetry has become a vital tool in treating patients in both outpatient, clinical and surgical settings.

A conventional pulse oximeter measures the variable electromagnetic energy absorption caused by blood volume changes. Pulse oximeters transmit electromagnetic energy at two different wavelengths, for example at 660 nm (red) and 940 nm (infrared, hereinafter IR) into the tissue and measure the attenuation of the energy as a function of time. The output signal of a pulse oximeter is sensitive to the pulsatile portion of the arterial blood flow. The output signal also contains a component that is a waveform representative of the patient's arterial pulse, sometimes referred to as a plethysmographic waveform or plethysmogram.

The period of rhythmic contraction of the heart by which blood is driven through the aorta and pulmonary artery is known as systole. Maximum light absorbance occurs during the systole of a cardiac cycle and is indicated on a plethysmogram by a low point or systolic valley. Conversely, the period of rhythmic relaxation and dilation of the heart cavities occurs during diastole when blood is drawn into the heart cavities. Minimum light absorbance occurs during the diastole of a cardiac cycle and is indicated on a plethysmogram by a high point or diastolic peak.

Pulse oximetry measurements typically use a digit, such as a finger, or an ear lobe or other element of the body, where blood flows close to the skin as the medium through which light energy is transmitted. The finger, for example, is composed of various tissues and substances including skin, fat, bone, muscle, blood, etc. The extent to which each of these biological tissues and substances attenuate incident electromagnetic energy is generally known. Look-up tables are often used to correlate the attenuation of the electromagnetic energy to pulsatile blood oxygen concentration, SpO₂.

A typical pulse oximeter may include a sensor, cabling from the sensor to a computer for signal processing and visual display, the computer and visual display typically being included in a patient monitor. The sensor typically includes two light emitting diodes (LEDs) placed across a finger tip and a photodetector on the side opposite the LEDs. The detector measures both transmitted light signals once they have passed through the finger. The signals are routed to a computer for analysis and display of the various parameters measured.

The two LEDs emit narrowband light (i.e., half power bandwidth of typically 15 nm) at two different frequency bands, typically red (centered at about 660 nm) and IR (centered at about 940 nm). The intensity of light transmitted through tissue, I_{transmitted}, is different for each wavelength of light emitted by the LEDs. Oxyhemoglobin (oxygenated blood) tends to absorb IR light, whereas deoxyhemoglobin (deoxygenated blood) tends to absorb red light. Thus, the absorption of IR light relative to the red light increases with oxyhemoglobin. The ratio of the absorption coefficients can be used to determine the oxygen saturation of the blood.

Portable pulse oximeters are well known and typically include a sensor that is left on the patient long enough for the oximeter to pick up a constant reading. Once the heart rate and oxygen saturation level have been measured and recorded, the sensor may be removed. Such conventional portable oximeters simply display the patient parameters or “data” on a small screen.

Pulse oximeters that are used for more long-term applications are typically capable of recording substantial amounts of data and allow for measurement of trends in the data. Some pulse oximeters are also configured for connection to a local area network. Such “network capable” pulse oximeters allow remote monitoring of patients. Network capable pulse oximeters may have a cable for connecting to the network. Alternatively, some network capable pulse oximeters may have a wireless “add-on” module to eliminate the need for cables. However, conventional wireless pulse oximeters do not appear to disclose formatting pulse oximetry and any other form of bodily parameter for display as a Web page. Accordingly, there exists a need in the art for a wireless pulse oximeter, a system for remote patient monitoring and a method for wirelessly transmitting pulse oximetry data.

### SUMMARY OF THE INVENTION

An embodiment of a wireless pulse oximeter according to the present invention may include a wireless sensor input for receiving raw pulse oximetry data and processor in communication with the wireless sensor. The processor may be configured for processing the raw pulse oximetry data to obtain processed pulse oximetry data and further configured to generate a Web page for displaying the processed pulse oximetry data. The wireless pulse oximeter may further include a wireless transceiver in communication with the processor and may be configured for communicating oximetry information including any of the raw pulse oximetry data, the processed pulse oximetry data and the Web page.

Additional features and advantages of the invention will be apparent from the detailed description which follows, taken in conjunction with the accompanying drawings, which together illustrate, by way of example, features of embodiments of the present invention.

### DESCRIPTION OF THE DRAWINGS

The following drawings illustrate exemplary embodiments for carrying out the invention. Like reference numerals refer to like parts in different views or embodiments of the present invention in the drawings:

FIG. 1 is a block diagram of a wireless pulse oximeter system according to an embodiment of the present invention.

FIG. 2 is a block diagram of a patient monitoring system according to an embodiment of the present invention.

FIG. 3 is a flow chart of a method for wirelessly transmitting pulse oximetry data according to an embodiment of the present invention.

FIG. 4 is a block diagram of a computer media for storing a computer program configured for implementing a method of wirelessly transmitting pulse oximetry data according to the present invention.

### DETAILED DESCRIPTION OF THE INVENTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

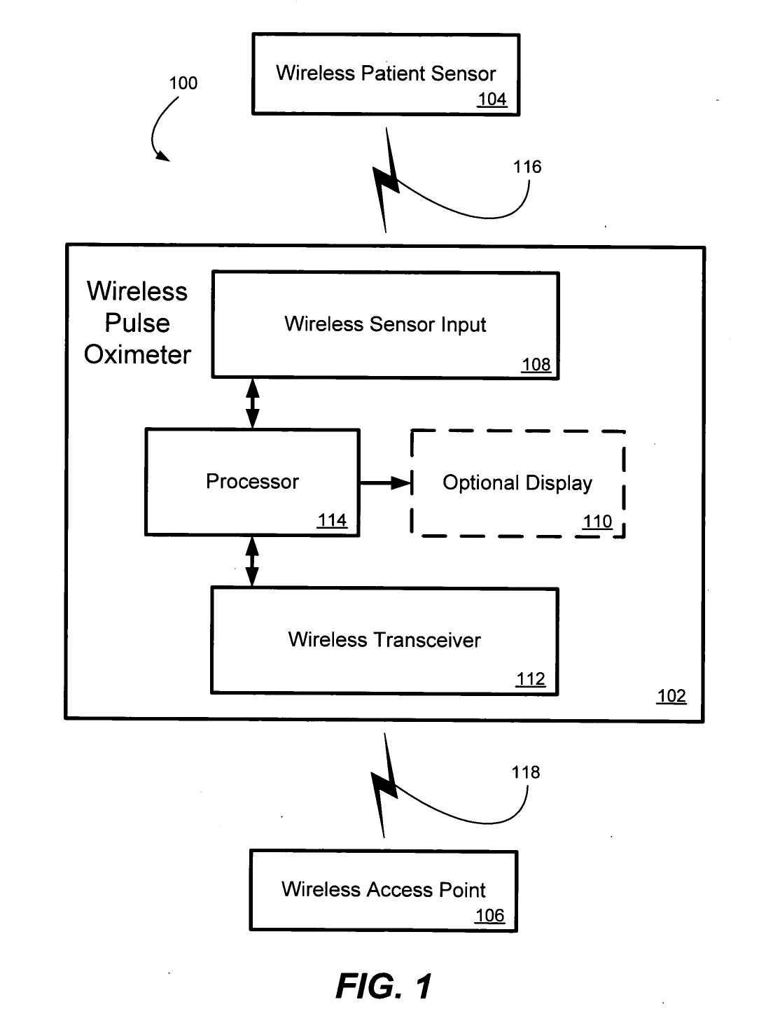

FIG. 1 is a block diagram of a wireless pulse oximeter system 100 according to an embodiment of the present invention. Wireless pulse oximeter system 100 may include a wireless pulse oximeter 102 configured for communication with a wireless patient sensor 104. According to another embodiment of the present invention, wireless pulse oximeter system 100 may further include a wireless access point 106 configured for communication with the wireless pulse oximeter 102. Wireless access point 106 may be configured for receiving information from wireless pulse oximeter 102 and providing access to raw or processed pulse oximetry information on a network.

Wireless patient sensor 104 may be configured for measuring raw pulse oximetry data from a patient and transmitting the raw pulse oximetry data to the wireless pulse oximeter 102. According to an embodiment of the present invention, wireless patient sensor 104 may further include red and infrared (IR) light sources (not shown) configured for transmitting red and IR light into patient tissue (not shown) and a light sensor (not shown) configured to receive attenuated transmitted or reflected light from the patient tissue and generate raw pulse oximetry data. Red and IR light sources and light sensors are well known to those skilled in the art and, thus, will not be elaborated on herein. Wireless patient sensor 104 may further include a transmitter (not shown) for transmitting the raw pulse oximetry data to the wireless pulse oximeter 102.

The transmitter included in the wireless patient sensor 104 may be configured for wireless communication 116 using any suitable transmission protocol or standard. For example and not by way of limitation, the transmitter may include at least one of the following wireless standards, protocols or technologies: Institute of Electrical and Electronics Engineers (IEEE) 802.11.a, IEEE 802.11.b, IEEE 802.11.g, Federal Communication Commission (FCC) Wireless Medical Telemetry Band (WMTS), infrared (IR), radio frequency (RF) transmission and Bluetooth®. Such transmitters suitable for use with the wireless patient sensor 104 are known to those skilled in the art and, thus, will not be further elaborated on herein.

Wireless pulse oximeter 102 may include a wireless sensor input 108, an optional display 110, a wireless transceiver 112 and a processor 114 in communication with the wireless sensor input 108, the optional display 110 and the wireless transceiver 112 according to an embodiment of the present invention. Wireless pulse oximeter 102 may also be configured with a battery (not shown for clarity) and/or suitable power supply (also not shown for clarity) for operating from a U.S. or European standard alternating current (A/C) outlet. Suitable batteries and/or power supplies are within the knowledge of those skilled in the art and, thus, will not be further elaborated on herein.

Optional display 110 may be configured for dynamically displaying various parameters, e.g., pulsatile blood oxygen concentration (SpO₂), heart rate, battery strength, wireless LAN signal strength, patient sensor signal strength, alarms, user settings and any other suitable parameters. Optional display 110 may be configured for displaying a Web page according to another embodiment of the present invention. Optional display 110 may include any suitable display technology including, but not limited to: liquid crystal display (LCD), light emitting diode (LED) display, organic LED (OLED) display, a polymer light emitting device (PLED) display, an electroluminescent (EL) display, an electrophoretic display, electrochromic display, electrowetting display, gas plasma display and a fiber plasma display. Such display technologies and their application are within the knowledge of one skilled in the art and, thus, will not be further elaborated on herein.

Wireless sensor input 108 may be configured for receiving raw pulse oximetry data from, for example and not by way of limitation, the wireless patient sensor 104. Wireless sensor input 108 will, of course, be compatible with the wireless transmission standard, as described above, for communicating 116 with the wireless patient sensor 104. Wireless sensor input 108 may be configured for receiving other types of bodily parameter signals, for example, processed pulse oximetry data, EKG, temperature and the like, according to other embodiments of the present invention.

Processor 114 may be configured for processing the raw pulse oximetry data to obtain processed pulse oximetry data. According to other embodiments of the present invention, processor 114 may be configured for processing processed pulse oximetry data, EKG data and temperature data. Processor 114 may further be configured to generate a Web page for displaying the processed pulse oximetry data and/or other bodily parameters. Wireless transceiver 112 may be configured for communicating oximetry information including any of the raw pulse oximetry data, processed pulse oximetry data, other bodily parameter data and the Web page. According to another embodiment of the present invention, the Web page may be the vehicle for transporting data, e.g., raw pulse oximetry data, processed pulse oximetry data, other bodily parameter data. According to another embodiment of wireless pulse oximeter system 100, wireless pulse oximeter 102 may also be configured for communication with a wireless access point 106 according to another embodiment of the present invention.

According to another embodiment of the present invention, wireless pulse oximeter 102 may further include memory (not shown) in communication with processor 114 for storing computer programs consistent with the method 300 (see below) of the present invention. The memory may further be configured for storing patient data such as raw and processed pulse oximetry data, EKG and temperature consistent with the present invention. The memory may be, for example and without limitation, a random access memory (RAM), a static RAM (SRAM), a dynamic RAM (DRAM), a magnetic RAM (MRAM), a nonvolatile electrically block-erasable programmable read only memory (FLASH) or any other suitable solid-state, mechanical or other memory capable of storing data and computer programs for use with processor 114.

Another embodiment of the present invention is a standalone wireless pulse oximeter. The standalone wireless pulse oximeter may include a wireless patient sensor configured for measuring and wirelessly transmitting raw pulse oximetry data from a patient and a wireless sensor input for receiving the raw pulse oximetry data. The standalone wireless pulse oximeter may further include a processor connected to the wireless sensor and configured for processing the raw pulse oximetry data to obtain processed pulse oximetry data and further configured to format the processed pulse oximetry data in a Web page. The standalone wireless pulse oximeter may further include a display in communication with the processor for displaying the Web page. Embodiments of the wireless patient sensor may be configured to transmit raw pulse oximetry data using any one of various suitable wireless transmission protocols or standards, e.g., Bluetooth, infrared and IEEE 802.11 in all of its variants (a, b and g). The Web page may comprise hypertext markup language (HTML) or other suitable formatting.

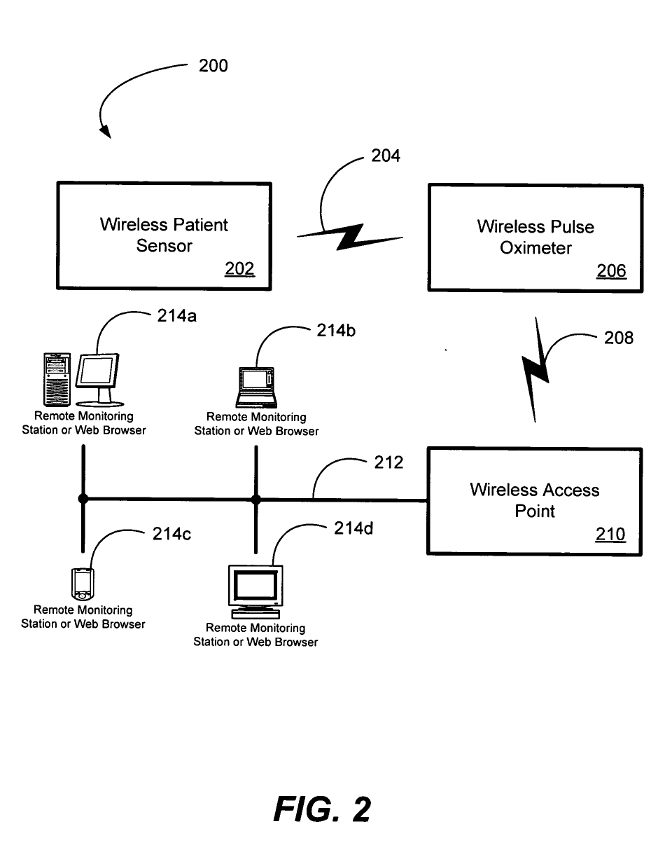

FIG. 2 is a block diagram of a patient monitoring system 200 according to an embodiment of the present invention. An embodiment of system 200 may include a wireless patient sensor 202 in communication 204 with a wireless pulse oximeter 206 in communication 208 with a wireless access point 210. Wireless access point 210 may further be connected to a network 212. Network 212 may be in communication with a plurality of remote monitoring stations 214a-d according other embodiments of system 200. Each remote monitoring stations 214a-d may be a personal computer (desktop 214d or laptop 214b), a server 214a, a personal digital assistant 214c or any other computing device with network access, computing capability and a web browser.

Wireless patient sensor 202 may be configured for measuring and transmitting raw pulse oximetry data from a patient as described above with respect to wireless patient sensor 104. Wireless patient sensor 202 may further be configured to measure and transmit a bodily signal representing other physical parameters such as electrocardiogram (EKG), temperature or any other bodily parameter that may be useful in determining the condition of a patient. Alternatively, wireless patient sensor 202 may comprise a plurality of sensors each dedicated to measuring and transmitting a particular bodily parameter according other embodiments of the present invention.

Wireless pulse oximeter 206 may be configured to receive and transmit the raw pulse oximetry data or other bodily signal from the wireless patient sensor 202. Wireless pulse oximeter 206 may be further configured to process and transmit the raw pulse oximetry data or other bodily signals as processed pulse oximetry data or processed bodily signals. Wireless pulse oximeter 206 may be further configured to process, format and transmit the processed pulse oximetry data or processed bodily signals for viewing as a Web page.

Wireless access point 210 may be configured for receiving and providing access to the raw pulse oximetry data, the processed pulse oximetry data or the Web page on a network according to an embodiment of the present invention. Remote monitoring stations 214a-d may be configured for communication with the network 212 and configured for receiving the raw pulse oximetry data, the processed pulse oximetry data or the Web page. Remote monitoring stations 214a-d may be configured with a processor to manipulate the raw pulse oximetry data, processed pulse oximetry data or the Web page. Remote monitoring stations 214a-d may be further configured with a display for displaying a Web page or for displaying the raw pulse oximetry data, processed pulse oximetry data in other formats, including but not limited to tables and graphics. Remote monitoring stations 214a-d may further be configured to process the raw pulse oximetry data and may further be configured to format and display the processed pulse oximetry data for viewing as a Web page, tables or graphics. The Web page, tables or graphics may be used to manage multiple patients either locally or remotely according to embodiments of the present invention. A Web page may itself be used to transmit raw pulse oximetry or other bodily parameter data for processing by another computer, i.e., a remote monitoring station, according to another embodiment of the present invention.

Wireless pulse oximeter 206 may include a wireless sensor input 108 configured for receiving the raw pulse oximetry data or any other bodily parameter signal from the wireless patient sensor 202 according to embodiments of the present invention. Wireless pulse oximeter 206 may further include a processor 114 in communication with the wireless sensor input 108 and configured for processing the raw pulse oximetry data to obtain the processed pulse oximetry data and further configured to format the processed pulse oximetry data for viewing as a Web page, tables or graphics. Wireless pulse oximeter 206 may further include a wireless transceiver 112 in communication with the processor 114 and may be configured for wirelessly communicating the raw pulse oximetry data, the processed pulse oximetry data, the Web page, tables or graphics.

Another embodiment of a wireless portable oximeter 102, 206 may include a hard-wired network port, e.g., Ethernet (not shown). Such a hard-wired network port may be useful for initially configuring the wireless pulse oximeter 102, 206, debugging, or for connection to a network that does not have wireless capabilities. Wireless portable oximeter 102, 206 may also be configured with one or more user controls, e.g., buttons, knobs, sliders, touch pads/screens and the like, for manipulating the operation of the device. Such user controls may, for example, but, not limited to adjusting display settings or alarms, measurement intervals, display parameters, network configuration and any other parameter, configuration or setting that may be appropriate for the wireless pulse oximeters 102, 206 described herein.

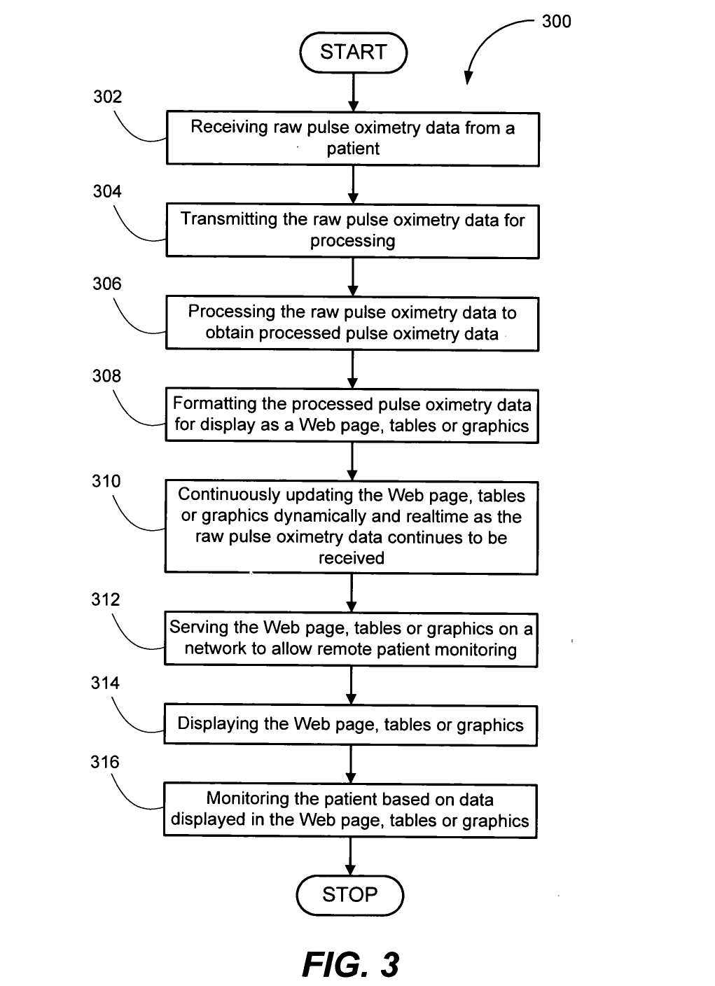

FIG. 3 is a flow chart of a method 300 for wirelessly transmitting pulse oximetry data according to an embodiment of the present invention. Method 300 may include receiving 302 raw pulse oximetry data from a patient and transmitting 304 the raw pulse oximetry data for processing. Method 300 may further include processing 306 the raw pulse oximetry data to obtain processed pulse oximetry data and formatting 308 the processed pulse oximetry data for display as a Web page, tables or graphics. Method 300 may further include continuously updating 310 the Web page, tables or graphics dynamically and in real-time as the raw pulse oximetry data continues to be received. Method 300 may further include serving 312 the Web page on a network to allow remote patient monitoring. Method 300 may further include displaying 314 the Web page, tables or graphics. Method 300 may further include monitoring 316 the patient based on the Web page, tables or graphics.

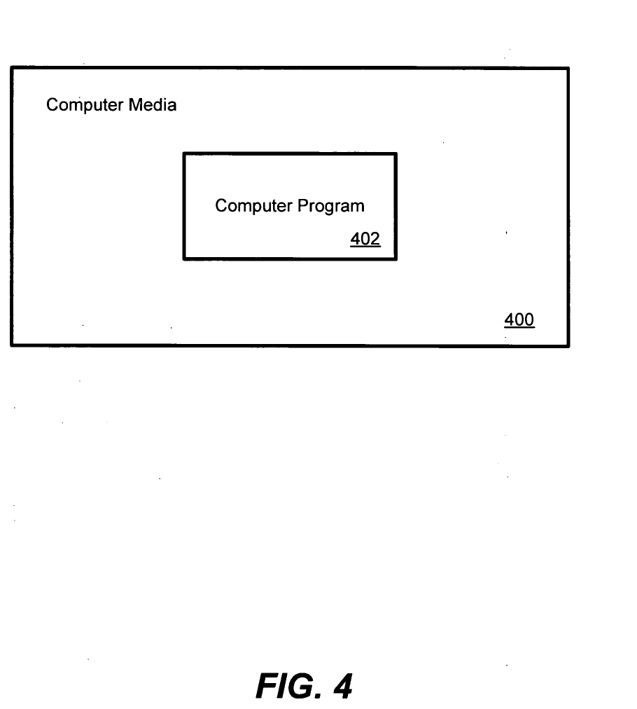

FIG. 4 is a block diagram of a computer media 400 for storing a computer program 402 configured for implementing the method 300 of wirelessly transmitting pulse oximetry data according to the present invention. Computer media 400 may be any suitable storage medium for storing a computer program 402, e.g., compact disc (CD), mini-disc (MD), read only memory (ROM), programmable ROM (PROM), electrically erasable PROM (EEPROM), nonvolatile electrically block-erasable programmable read only memory (FLASH), or any other suitable media for storing a computer program 402. Flash memory for storing a computer program 402 may take many forms, for example and not by way of limitation, Memory Stick™ or SD memory card™ that plugs into a housing (not shown) of the wireless pulse oximeter 102, 206. Computer media 400 may also be firmware embedded in an operating system of a wireless pulse oximeter 102, 206, or an applet or plug-in down-loadable and configurable computer program, or device driver for execution on the wireless pulse oximeter 102, 206. The particular type of computer media 400 and the form of the computer program 402 are not critical to the present invention.

The above embodiments have been described with reference to a wireless pulse oximeter system 100 and patient monitoring system 200 for use in measuring pulse oximetry data, EKG, temperature and other bodily parameters of a patient. However, systems 100 and 200 are not limited to measuring a single patient at a time. Embodiments of a wireless pulse oximeter system 100 and patient monitoring system 200 may also be configured to measure, process, transmit and display bodily parameters of a plurality of patients simultaneously using various schemes, for example and not limited to time division multiple access (TDMA), code division multiple access (CDMA), frequency-hopping spread spectrum (FHSS) or direct-sequence spread spectrum (DSSS) technologies as known to one of skill in the art.

Although this invention has been described with reference to particular embodiments, the invention is not limited to these described embodiments. Rather, the invention is limited only by the appended claims, which include within their scope all equivalent devices or methods that operate according to the principles of the invention as described herein.

1. A wireless pulse oximeter, comprising:
a wireless sensor input for receiving raw pulse oximetry data;
a processor in communication with the wireless sensor configured for processing the raw pulse oximetry data to obtain processed pulse oximetry data and further configured to generate a Web page for displaying the processed pulse oximetry data; and
a wireless transceiver in communication with the processor and configured for communicating oximetry information including any of the raw pulse oximetry data, the processed pulse oximetry data and the Web page.

2. The wireless pulse oximeter according to claim 1, wherein the wireless sensor input is further configured to receive signals representative of at least one of electrocardiogram (EKG) and temperature.

3. The wireless pulse oximeter according to claim 1, further comprising a wireless patient sensor configured for measuring the raw pulse oximetry data and transmitting the raw pulse oximetry data to the wireless sensor input.

4. The wireless pulse oximeter according to claim 3, wherein the wireless patient sensor further comprises:
red and infrared (IR) light sources configured for transmitting red and IR light into patient tissue;
a light sensor configured to receive attenuated transmitted or reflected light from the patient tissue and generate the raw pulse oximetry data; and
a transmitter for transmitting the raw pulse oximetry data to the wireless sensor input.

5. The wireless pulse oximeter according to claim 4, wherein the transmitter incorporates at least one of the following wireless standards, protocols or technologies:
Institute of Electrical and Electronics Engineers (IEEE) 802.11a, IEEE 802.11b, IEEE 802.11g, Federal Communication Commission (FCC) Wireless Medical Telemetry Band (WMTS), infrared (IR), radio frequency (RF) transmission and Bluetooth®.

6. The wireless pulse oximeter according to claim 1, further comprising a display configured for displaying parameters including at least one of pulsatile blood oxygen concentration (SpO₂), heart rate, battery strength, wireless LAN signal strength, patient sensor signal strength, alarms and user settings.

7. The wireless pulse oximeter according to claim 6, wherein the display is further configured to display the parameters in the Web page.

8. The wireless pulse oximeter according to claim 1, further comprising a wireless access point for receiving information from the wireless transceiver and providing access to the oximetry information on a network.

9. A standalone wireless pulse oximeter, comprising:
a wireless patient sensor configured for measuring and wirelessly transmitting raw pulse oximetry data from a patient;
a wireless sensor input for receiving the raw pulse oximetry data; and
a processor connected to the wireless sensor and configured for processing the raw pulse oximetry data to obtain processed pulse oximetry data and further configured to format the processed pulse oximetry data in a Web page.

10. The standalone wireless pulse oximeter according to claim 9, further comprising a display in communication with the processor for displaying the Web page.

11. The standalone wireless pulse oximeter according to claim 9, wherein the wireless patient sensor wirelessly transmits the raw pulse oximetry data using at least one of Bluetooth, infrared, radio frequency, and IEEE 802.11 wireless transmission standards.

12. The standalone wireless pulse oximeter according to claim 9, wherein the Web page comprises hypertext markup language (HTML).

13. A patient monitoring system, comprising:
a wireless patient sensor configured for measuring and transmitting raw pulse oximetry data from a patient;
a wireless pulse oximeter configured to receive the raw pulse oximetry data from the wireless patient sensor and configured to transmit the raw pulse oximetry data and configured to process and transmit the raw pulse oximetry data as processed pulse oximetry data and configured to format and transmit the processed pulse oximetry data for viewing as a Web page, tables or graphics;
a wireless access point configured for receiving and providing access to the raw pulse oximetry data, the processed pulse oximetry data or the Web page, tables or graphics on a network; and
a remote monitoring station in communication with the network configured for receiving the raw pulse oximetry data, the processed pulse oximetry data or the Web page, tables or graphics.

14. The patient monitoring system according to claim 13, wherein the wireless pulse oximeter comprises:
a wireless sensor input configured for receiving the raw pulse oximetry data from the wireless patient sensor;
a processor in communication with the wireless sensor input and configured to process the raw pulse oximetry data as processed pulse oximetry data and configured to format the processed pulse oximetry data for viewing as a Web page, tables or graphics; and
a wireless transceiver in communication with the processor and configured for wirelessly transmitting the raw pulse oximetry data, the processed pulse oximetry data, the Web page, tables or graphics.

15. The patient monitoring system according to claim 13, wherein the remote computer is further configured to process the raw pulse oximetry data and further configured to format and display the processed pulse oximetry data for viewing as a Web page.

16. A method for wirelessly transmitting pulse oximetry data, comprising:
receiving raw pulse oximetry data from a patient;
transmitting the raw pulse oximetry data for processing;
processing the raw pulse oximetry data to obtain processed pulse oximetry data;
formatting the processed pulse oximetry data for display as a Web page; and
continuously updating the Web page dynamically and in real-time as the raw pulse oximetry data continues to be received.

17. The method according to claim 16, further comprising serving the Web page on a network to allow remote patient monitoring.

18. The method according to claim 16, further comprising displaying the Web page, tables or graphics.

19. The method according to claim 16, further comprising monitoring the patient based on the Web page, tables or graphics.

20. A wireless pulse oximeter, comprising:
a means for wirelessly receiving raw pulse oximetry data;
a means for processing the raw pulse oximetry data to obtain processed pulse oximetry data; and
a means for formatting the processed pulse oximetry data as a Web page.

21. The wireless pulse oximeter according to claim 20, further comprising a means for displaying the Web page.

22. The wireless pulse oximeter according to claim 20, further comprising a means for serving the Web page on a network.

23. The wireless pulse oximeter according to claim 20, wherein the means for processing the raw pulse oximetry data comprises an oximetry module.

24. The wireless pulse oximeter according to claim 20, wherein the means for formatting the processed pulse oximetry data comprises at least one of server-side includes (SSI), Javascript, Common Gateway Interface (CGI), Active Server Pages (ASP), PHP: Hypertext Preprocessor (PHP) and Extensible Markup Language (XML).

25. A computer media for storing a computer program, the computer program implementing a method of wirelessly transmitting pulse oximetry data, the method comprising:
receiving raw pulse oximetry data from a patient;
transmitting the raw pulse oximetry data for processing;
processing the raw pulse oximetry data to obtain processed pulse oximetry data;
formatting the processed pulse oximetry data for display as a Web page; and
continuously updating the Web page dynamically and in real-time as the raw pulse oximetry data continues to be received.
